(19) United States
(12) Patent Application Publication
(10) Pub. No.: US 2016/0045172 A1
(43) Pub. Date: Feb. 18, 2016

(54) ACTIVITY METER AND EVENT INFORMATION RECORDING SYSTEM

(71) Applicant: Kabushiki Kaisha Toshiba, Minato-ku (JP)

(72) Inventor: Wataru ARATANI, Kawasaki (JP)

(73) Assignee: Kabushiki Kaisha Toshiba, Minato-ku (JP)

(21) Appl. No.: 14/800,193

(22) Filed: Jul. 15, 2015

(30) Foreign Application Priority Data
Aug. 18, 2014 (JP) 2014-165791

(51) Int. Cl. A61B 5/00; A61B 5/11

(52) U.S. Cl. CPC A61B 5/7475; A61B 5/1118; A61B 5/681; A61B 5/0015; A61B 2562/0219; A61B 2560/0406; A61B 2560/0475

(57) ABSTRACT

According to an embodiment, an activity meter registers, based on an acceleration sensor and an output signal of the acceleration sensor when a tapping operation is performed, information on the tapping operation, detects generation of a tapping operation identical to the tapping operation corresponding to the registered information from the output signal of the acceleration sensor, and stores, when generation of the tapping operation identical to the tapping operation corresponding to the registered information is detected, time information when the identical tapping operation is generated in a memory.

### CROSS-REFERENCE TO RELATED APPLICATION

This application is based upon and claims the benefit of priority from the prior Japanese Patent Application No. 2014-165791 filed in Japan on Aug. 18, 2014; the entire contents of which are incorporated herein by reference.

### FIELD

An embodiment described herein relates generally to an activity meter and an event information recording system.

### BACKGROUND

Activity meters that measure amounts of human activity are becoming widespread in recent years. Conventionally, there are proposals of devices that record biological data such as cardiogram data and human actions.

Among these devices, there are ones worn on a human arm or a belt to detect human motion using an acceleration sensor and determine the presence or absence of predetermined human motion based on an output value of the acceleration sensor.

There is also a proposal of a body motion detection apparatus capable of detecting attachment/detachment of a mounting body attachable/detachable to/from a main unit from an acceleration change caused by an impact added to an impact protrusion of an exercise amount meter.

However, conventional pedometers or these proposed devices determine the presence or absence of predetermined motion from an output of an acceleration sensor, but these devices cannot accurately determine all actions of users.

Moreover, people may want to record actions and events in their lives, and record the actions and events in a memo pad, smartphone or the like. However, it is complicated to write the actions and events into a memo pad or input data to a smartphone or the like.

### BRIEF DESCRIPTION OF THE DRAWINGS

FIG. 1 is an outline view of a wristband-type activity meter 1 according to an embodiment;

FIG. 2 is a configuration diagram of an event recording system 100 made up of the wristband-type activity meter and a smartphone according to the embodiment;

FIG. 3 is a block diagram illustrating a configuration of the activity meter 1 according to the embodiment;

FIG. 4 is a flowchart illustrating an example of a tap count registering process according to the embodiment;

FIG. 5 is a flowchart illustrating another example of the tap count registering process according to the embodiment;

FIG. 6 is a diagram illustrating a configuration example of a table TBL1 according to the embodiment;

FIG. 7 is a flowchart illustrating an example of an event information recording process when event information is recorded in the activity meter 1 by a user's tapping operation according to the embodiment;

FIG. 8 is a flowchart illustrating another example of the event information recording process when event information is recorded in the activity meter 1 by the user's tapping operation according to the embodiment;

FIG. 9 is a diagram illustrating a configuration example of an event information table TBL2 according to the embodiment;

FIG. 10 illustrates a configuration example of a correspondence table TBL3 which registers a correspondence between tap counts and events according to the embodiment; and

FIG. 11 is a display example of an event recording information list TBL4 displayed on a display section 2a according to the embodiment.

### DETAILED DESCRIPTION

An activity meter of an embodiment includes a tapping operation detection section configured to detect, when a tapping operation is performed, information on the tapping operation, a tapping operation information registration section configured to register the information on the tapping operation based on detected information from the tapping operation detection section, a tapping operation generation detection section configured to detect generation of a tapping operation identical to the tapping operation corresponding to the registered information from the detected signal of the tapping operation detection section, and an identical tapping operation output section configured to output, when the tapping operation generation detection section detects generation of the tapping operation identical to the tapping operation corresponding to the registered information, information indicating that the identical tapping operation is generated.

An activity meter of the embodiment includes an acceleration sensor, a tapping operation information registration section configured to register, based on an output signal of the acceleration sensor when a tapping operation is performed, information on the tapping operation, a tapping operation generation detection section configured to detect generation of a tapping operation identical to the tapping operation corresponding to the registered information from an output signal of the acceleration sensor, and a time information storage section configured to store, when the tapping operation generation detection section detects generation of the tapping operation identical to the tapping operation corresponding to the registered information, time information when the identical tapping operation is generated.

An event information recording system according to the embodiment made up of an activity meter wearable on a target person whose activity amount is measured and a terminal communicable with the activity meter, the activity meter including an acceleration sensor, a tapping operation information registration section configured to register, based on an output signal of the acceleration sensor when a tapping operation is performed, information on the tapping operation, a tapping operation generation detection section configured to detect generation of a tapping operation identical to the tapping operation corresponding to the registered information from an output signal of the acceleration sensor, a time information storage section configured to store, when the tapping operation generation detection section detects generation of the tapping operation identical to the tapping operation corresponding to the registered information, time information when the identical tapping operation is generated, and an information transmitting section configured to transmit the stored time information, and the terminal including a storage section configured to store the time information received from the activity meter in association with predetermined event information.

### Embodiment

Hereinafter, an embodiment will be described with reference to the accompanying drawings.

### Configuration

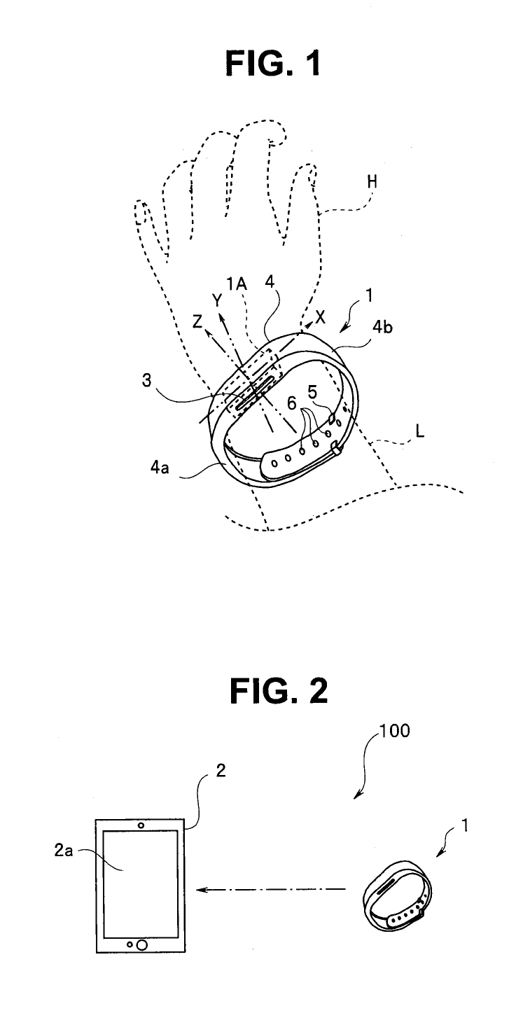

FIG. 1 is an outline view of a wristband-type activity meter 1 according to an embodiment. FIG. 2 is a configuration diagram of an event information recording system 100 made up of the wristband-type activity meter and a smartphone.

The activity meter 1 wearable on a target person whose activity amount is measured is a wristband-type device capable of recording an activity amount of the user and transmitting the activity amount to a smartphone 2.

The activity meter 1 is a band-shaped wristband type to be worn by being wrapped around the user's arm (shown by a dotted line) L and an elongated operation button 3 is provided in a center thereof. The operation button 3 is operated when making various settings or the like.

The activity meter 1 includes two extended portions 4a and 4b that extend from both sides of a central portion of a band 4 on which the operation button 3 or the like is arranged. The band 4 is a case of the activity meter 1. A clasp 5 is provided at an end portion of the extended portion 4a and a plurality of holes 6 into which a protruding portion (not shown) of the clasp 5 is fitted are formed in the extended portion 4b at a predetermined interval. The user can wear the activity meter 1 on the arm L by fitting the protruding portion (not shown) formed in the clasp 5 into the hole 6 at an arbitrary position.

The operation button 3 is disposed in the activity meter 1 so as to come to one side face of the activity meter 1 in close contact with the arm L when the user wears the activity meter 1 on the arm L as shown in FIG. 1.

Note that although the embodiment shown in FIG. 1 shows an example of the position of the operation button 3 where the operation button 3 is attached to one side face of the activity meter 1, without being limited to the example, the operation button 3 may be provided so as to be located on a top surface side (that is, front side) of the activity meter 1 (band 4 or main unit 1A) in close contact with the arm L.

The event information recording system 100 shown in FIG. 2 is an event information recording system using the activity meter 1.

As will be described later, the activity meter 1 has a radio communication function and can transmit event information which will be described later to the smartphone 2. The user can transmit data such as event information recorded in the activity meter 1 to the smartphone 2, store the data in a memory of the smartphone 2, and manage the data according to an application program stored in the smartphone 2 or display the data on a display section 2a of the smartphone 2.

Note that the activity meter 1 has an acceleration sensor, generates, from an output of the acceleration sensor, records and transmits activity data such as the user's activity amount and a count of steps. Here, description of recording and transmission of activity data is omitted and a recording and transmission process of event information which is one of the functions of the activity meter 1 will be described.

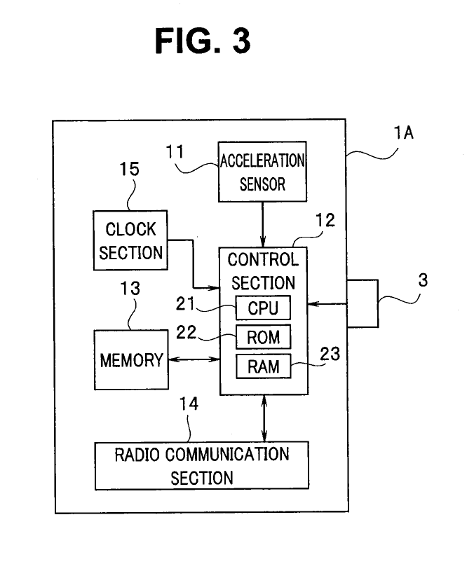

FIG. 3 is a block diagram illustrating a configuration of the activity meter 1 according to the present embodiment.

The activity meter 1 has a main unit 1A which is one unit including an acceleration sensor 11, a control section 12, a memory 13, a radio communication section 14 and a clock section 15. As shown by a dotted line in FIG. 1, the main unit 1A is an internal core detachably attached to the central portion of the band 4.

That is, the band 4 which is a wristband is a case configured to incorporate the acceleration sensor 11 or the like and the main unit 1A which is a unit is attachable/detachable to/from the band 4. The acceleration sensor 11 is a tapping operation detection section which detects, when a tapping operation is performed, information on the tapping operation, as will be described later.

The activity meter 1 has an activity amount measuring function, an event recording function which will be described later, a function of recording and transmitting the measured activity amount and a function of recording and transmitting the recorded event information.

The acceleration sensor 11 is a three-axis acceleration sensor which includes three sensors so as to be able to detect accelerations in three axis (X-axis, Y-axis and Z-axis) directions orthogonal to each other and outputs X-axis output, Y-axis output and Z-axis output as acceleration signals of the respective axes. Each output of the acceleration sensor 11 is inputted to the control section 12.

As shown in FIG. 1, when the activity meter 1 is worn on the user's arm L, the user can wear the activity meter 1 on the arm L so that the X-axis direction is parallel to a back of a hand H and orthogonal to an axis of the arm L, the Y-axis direction is parallel to the back of the hand H and parallel to the axis of the arm L, and the Z-axis direction is orthogonal to the back of the hand H. In FIG. 1, the arm L is passed through the ring-shaped band 4 of the activity meter 1 so that the arm L is oriented from the front toward the back of the sheet.

Note that the respective X-axis, Y-axis and Z-axis directions of the acceleration sensor 11 in the activity meter 1 are not limited to the directions shown in FIG. 1.

Note that the acceleration sensor 11 is a three-axis acceleration sensor here, but the acceleration sensor 11 may also be a 4- or more-axis acceleration sensor.

The control section 12 includes a central processing unit (hereinafter referred to as “CPU”) 21, a ROM 22, a RAM 23 and various interfaces (not shown) (hereinafter referred to as “I/F”).

The ROM 22 of the control section 12 stores, in addition to a program for measuring and recording an activity amount, a program for registering a tap count which will be described later, setting, recording and transmitting event information. Note that the ROM 22 may be a rewritable non-volatile memory such as a flash memory.

The CPU 21 can acquire an X-axis output, a Y-axis output and a Z-axis output of the acceleration sensor 11 via an I/F (not shown).

Furthermore, the CPU 21 can acquire an operating state of the operation button 3 via the I/F (not shown).

The radio communication section 14 is a circuit for carrying out data communication with the smartphone 2 and is a circuit for short-distance radio communication.

The clock section 15 is a circuit that generates and outputs time information, and the CPU 21 can acquire information on a date and a time from the clock section 15.

### Operation

First, a tap count registering process will be described.

For example, when the user wears the activity meter 1 on the arm, the user may want to record various events in life. Examples of events include having a meal, eating between meals or going to a lavatory.

As will be described hereinafter, when the user makes a registered number of single taps (that is, lightly tapping once; hereinafter referred to as “tap”) on the activity meter 1, the user can record the time at that moment in the memory 13 of the activity meter 1. Furthermore, the user can distinguish among events by the number of taps and record a plurality of desired evens in the activity meter 1. For example, the user may decide to use a one-time tap and a two-time tap to record events, and set the number of taps in the activity meter 1.

A correspondence between a tap count and an event is arbitrarily determined by the user and is not preset in the activity meter 1. As will be described later, the correspondence between a tap count and an event is set by the user using the smartphone 2. The user determines the correspondence between a tap count and an event, and sets only a tap count in the activity meter 1.

Thus, a certain user A may associate a one-time tap with an event of wake-up, associate a three-time tap with an event of having a meal, while another user B may associate a one-time tap with an event of wake-up, associate a two-time tap with an event of having a meal and associate a four-time tap with an event of going to bed. In that case, the one-time and three-time tap counts are registered in the activity meter 1 of the user A and the one-time, two-time and four-time tap counts are registered in the activity meter 1 of the user B.

Note that the aforementioned events are the users' actions, but may also be happenings or the like. An event can be, for example, a “happy event” or a “sad event.”

Thus, the user associates a desired event with an arbitrary tap count, sets and registers only the tap counts in the activity meter 1.

### Tap Count Registering Process

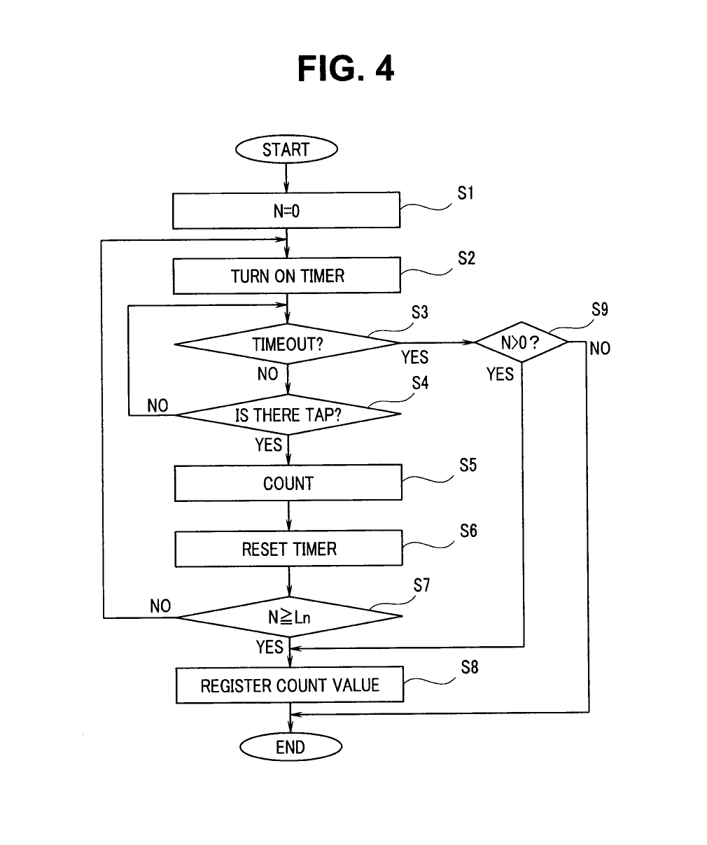

FIG. 4 is a flowchart illustrating a flow of a tap count registering process.

When the user performs a predetermined operation on the operation button 3, for example, a long pressing of the button, the CPU 21 reads a tap count registering processing program from the ROM 22 and executes the process in FIG. 4.

An output of the operation button 3 is inputted to the CPU 21 and upon detecting a predetermined operation on the operation button 3, the CPU 21 resets a counter value N to 0 (S1). The counter is a counter generated in the RAM 23.

After S1, the CPU 21 turns ON a timer (S2). This timer is, for example, a software timer and a time period until a timeout occurs, for example, 3 seconds, is preset.

After S2, the CPU 21 determines whether the timer which is turned ON in S12 times out or not (S3).

If the timer does not time out (S3: NO), the CPU 21 determines the presence or absence of a tap (S4). The presence or absence of a tap is determined based on an output value of an acceleration signal of the acceleration sensor 11. A tap is, for example, an operation for the user to lightly tap on and around a central portion of the band 4 of the activity meter 1 by his/her finger. The CPU 21 can determine the presence or absence of a tap based on an abrupt change in an output waveform of the acceleration sensor 11 at the time of such a tap.

When a tap is made (S4: YES), the CPU 21 performs counting (S5), or specifically, the CPU 21 increments the count value of the counter by 1.

After S5, the CPU 21 resets the timer (S6) and determines whether the counter value N is equal to or higher than a predetermined number Ln or not (S7). The predetermined number Ln is an upper limit value of the tap count.

When no tap is made (S4: NO), the process proceeds to S3.

When the counter value N is not equal to or higher than the predetermined number Ln (S7: NO), the process proceeds to S2.

When the timer times out (S3: YES), the CPU 21 determines whether the count value N is 0 or not (S9).

When the count value N is not 0 (S9: YES) and when the count value N is equal to or higher than the predetermined number Ln (S7: YES), the CPU 21 registers the count value N in a predetermined table TBL1 of the memory 13 (S8).

When the count value N is 0 (S9: NO), the process ends.

The tap count is registered as described above.

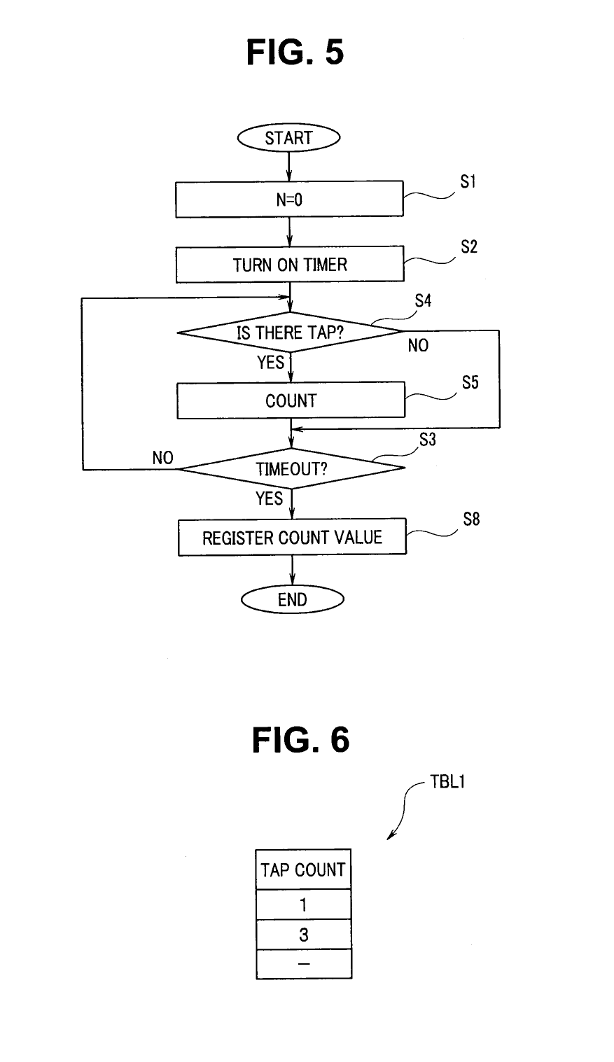

Note that the process in FIG. 4 may be substituted by a process in FIG. 5.

FIG. 5 is a flowchart illustrating another example of the tap count registering process. Note that in FIG. 5, the same processes as those in FIG. 4 are assigned the same step numbers (S) and description thereof will be simplified.

After the processes in S1 and S2, the CPU 21 determines the presence or absence of a tap (S4).

When a tap is made (S4: YES), the CPU 21 performs counting (S5). Specifically, after the timer turns ON (S2), if it is determined that a first tap is made, the CPU 21 performs counting that sets the count value of the counter in the RAM 23 to 1. When no tap is made (S4: NO), the process proceeds to S3.

When no tap is made (S4: NO) and after counting is performed (S5), the CPU 21 determines whether the timer times out or not (S3).

When the timer does not time out (S3: NO), the process returns to S4. When the timer times out (S3: YES), the CPU 21 registers the count value counted in S5 in the predetermined table TBL1 of the memory 13. Thus, the registering processing method in FIG. 5 is a method whereby counting is performed by the number of times taps are made after the timer turns ON until a timeout occurs.

FIG. 6 is a diagram illustrating a configuration example of the table TBL1. The table TBL1 is a tapping operation information registration table with which information on a tapping operation is registered and the information on the tapping operation is defined by a tap count. In the example of FIG. 6, information pieces on two tapping operations corresponding to a one-time tap and a three-time tap are set and registered.

Therefore, the processes in FIG. 4 and FIG. 5 constitute a tapping operation information registration section configured to register information on a tapping operation in the memory 13 based on an output signal of the acceleration sensor 11 when the tapping operation is performed.

Especially when an operation (a long pressing operation in the above example) on the operation button 3 is performed as a predetermined operation, information on the tapping operation is registered based on the output signal of the acceleration sensor 11 within a predetermined time period after the operation is performed. Through the processes in FIG. 4 or FIG. 5, a plurality of pieces of information on a plurality of mutually different tapping operations are registered in the table TBL1.

### Detection of Tapping Operation and Event Information recording Process

When information on a tapping operation is registered in the activity meter 1 through the processes in FIG. 4 or FIG. 5, an event information recording process when the user records an event will be described.

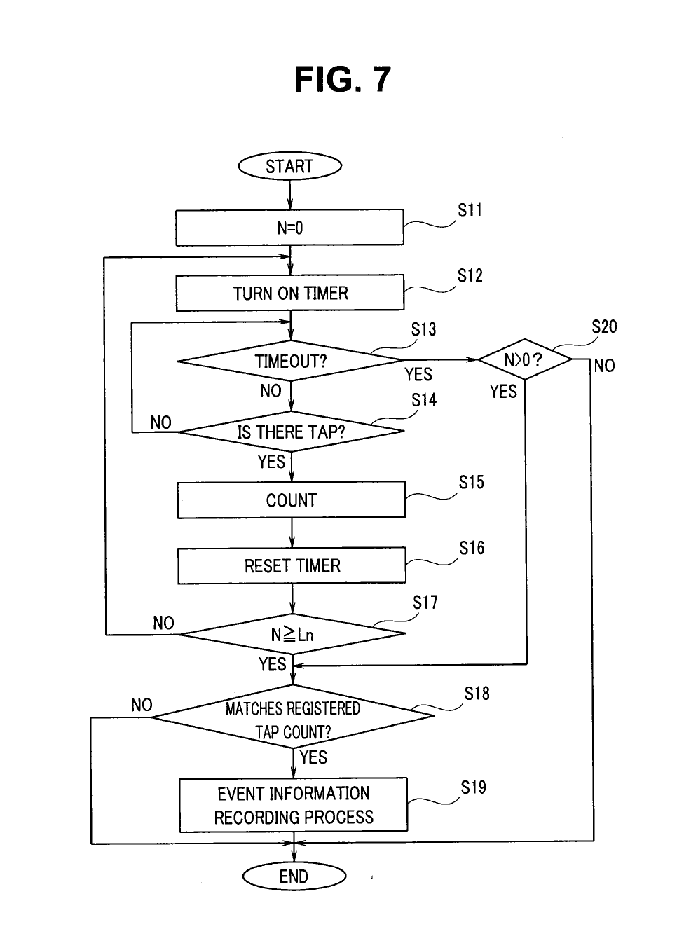

FIG. 7 is a flowchart illustrating an example of an event information recording process when event information is recorded in the activity meter 1 by the user's tapping operation.

When the user performs a predetermined operation, for example, a short pressing operation, on the operation button 3, the CPU 21 reads an event information recording processing program from the ROM 22 and executes the process in FIG. 7.

An output of the operation button 3 is inputted to the CPU 21, and upon detecting a predetermined operation on the operation button 3, the CPU 21 resets the value N of a counter to 0 (S11). The counter is generated in the RAM 23.

After S11, the CPU 21 turns ON a timer (S12). This timer is, for example, a software timer and a time period until a timeout occurs, for example, 2 seconds is preset.

After S12, the CPU 21 determines whether the timer which is turned ON in S12 times out or not (S13).

If the timer does not time out (S13: NO), the CPU 21 determines the presence or absence of a tap (S14). The presence or absence of a tap is determined based on an output value of an acceleration signal of the acceleration sensor 11, and the CPU 21 also determines the presence or absence of a tap based on an abrupt change in an output waveform of the acceleration sensor 11 here.

When a tap is made (S14: YES), the CPU 21 performs counting (S15), or specifically, the CPU 21 increments the count value of the counter by 1.

After S15, the CPU 21 resets the timer (S16), and determines whether the counter value N is equal to or higher than a predetermined number Ln which is an upper limit value of the tap count (S17).

When no tap is made (S14: NO), the process proceeds to S13.

When the count value N is not equal to or higher than the predetermined number Ln (S17: NO), the process proceeds to S12.

When the timer times out (S13: YES), the CPU 21 determines whether the count value N is 0 or not (S20).

When the count value N is not 0 (S20: YES), and when the count value N is equal to or higher than the predetermined number Ln (S17: YES), the CPU 21 determines whether the count value matches the registered tap count or not (S18). Specifically, the CPU 21 determines whether the count value counted in S15 is equal to one of tap counts registered in the table TBL1 in the memory 13 or not.

The processes from S11 to S18 constitute a tapping operation generation detection section configured to detect generation of a tap operation identical to the tap operation corresponding to the information on the tap operation registered in the table TBL1.

When the count value counted in S15 matches one of tap counts registered in the table TBL1 (S18: YES), the CPU 21 executes an event information recording process (S19).

When the count value counted in S15 does not match one of tap counts registered in the table TBL1 (S18: NO), and when the count value N is 0 (S20: NO), the process ends.

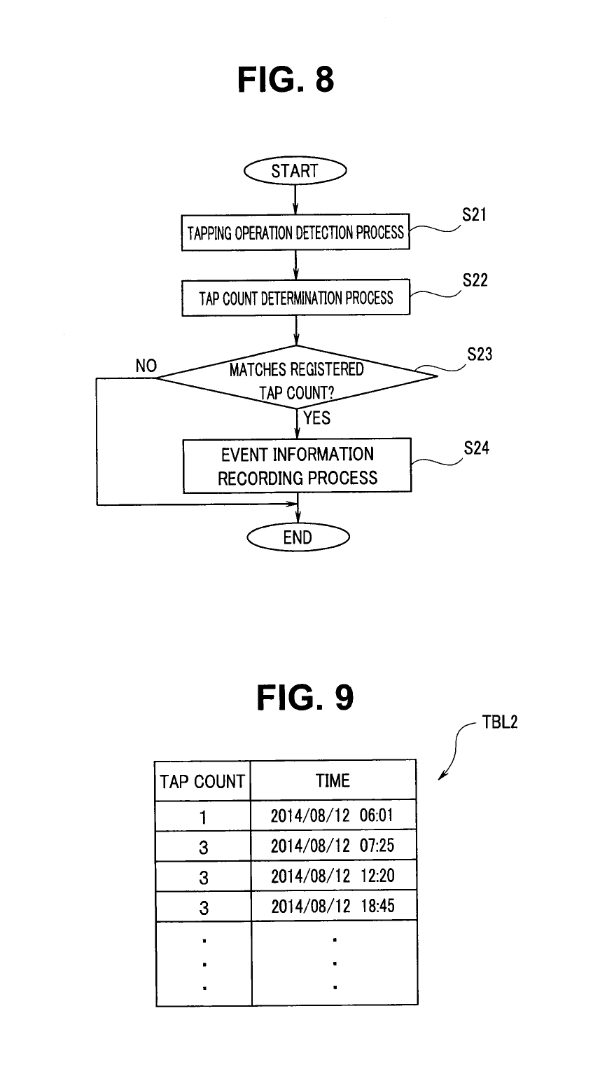

As described above, event information is registered. Note that the process in FIG. 7 may be substituted by a process in FIG. 8.

FIG. 8 is a flowchart illustrating another example of the event information recording process when event information is recorded in the activity meter 1 by the user's tapping operation.

When the user performs a predetermined operation on the operation button 3, for example, when the user presses the operation button 3 for a short time period, the process in FIG. 8 is executed.

First, the CPU 21 executes a tapping operation detection process (S21). The tapping operation detection process is a process that detects a tapping operation performed within a predetermined time period after the operation button 3 is pressed. The CPU 21 determines the presence or absence of a tapping operation based on the presence or absence of an abrupt change in an output waveform of the acceleration sensor 11.

After S21, the CPU 21 executes a tap count determination process (S22). In S22, the CPU 21 determines a tap count within a predetermined time period after the tapping operation detected in S21.

After S22, the CPU 21 determines whether the determined tap count matches the registered tap count or not (S23). Specifically, the CPU 21 determines whether the tap count determined in S22 is equal to one of tap counts registered in the table TBL1 in the memory 13 or not.

The processes from S21 to S23 constitute a tapping operation generation detection section configured to detect generation of a tapping operation identical to the tapping operation corresponding to information on the tapping operation registered in the table TBL1 from an output signal of the acceleration sensor 11.

When the determined tap count matches the registered tap count (S23: YES), the CPU 21 executes an event information recording process (S24). When the tap count determined in S22 is equal to one of tap counts registered in the table TBL1, the event information recording process is a process that records information on the tap count together with time information of the clock section 15 in an event information table TBL2 in the memory 13.

Note that since the timer is reset and turned ON for each tap in FIG. 4 and FIG. 7, even when the user wants to increase the number of taps, the user can perform tapping calmly and if the user adjusts the timer time appropriately, even when the number of taps is small, the user does not feel that the time period to confirm the input is too long.

As described above, when generation of the tapping operation identical to the tapping operation corresponding to information on the registered tapping operation is detected, the processes in S19 and S24 constitute a time information storage section configured to store time information when the identical tapping operation is generated in the event information table TBL2 of the memory 13.

Note that in the processes in S19 and S24, time information need not be stored in the event information table TBL2 in the memory, and in that case, when generation of the tapping operation identical to the registered tapping operation is detected, the processes in S19 and S24 constitute an identical tapping operation output section configured to output information (time information may not be included) indicating that the identical tapping operation is generated so as to be stored in the memory 13.

In the case of FIG. 6, for example, since one-time and three-time tap counts are registered in the table TBL1, when a tapping operation of one-time tap or three-time tap is performed in S23, the CPU 21 records information on the tap count together with the time information when the tapping operation is performed in the event information table TBL2.

FIG. 9 is a diagram illustrating a configuration example of the event information table TBL2. In the example in FIG. 9, the event information table TBL2 records information on a plurality of tap count/time pairs.

Note that in the processes in FIG. 7 and FIG. 8, when only one piece of information on the tapping operation is stored in the table TBL1, only time information may be recorded in the event information table TBL2.

As described above, when a predetermined operation is performed on the operation button (3), generation of the tapping operation identical to the tapping operation corresponding to the information on the tapping operation registered in the table TBL1 is detected, and when the generation of the identical tapping operation is detected, the event information is recorded in the event information table TBL2.

Note that when, for example, the operation button 3 is pressed for a short time period as a predetermined operation here, the process in FIG. 8 is executed, but even when the predetermined operation is not performed, the CPU 21 may monitor the output of the acceleration sensor 11 all the time and monitor whether a tapping operation is performed or not all the time to thereby detect generation of a tapping operation and register event information.

### Event Information Transmission Process

The CPU 21 transmits event information recorded in the event information table TBL2 from the radio communication section 14 to the smartphone 2 at predetermined timing. The predetermined timing may be timing in a preset time period in the activity meter 1 or timing of a transmission request from the smartphone 2.

Alternatively, when only a small free space is left in the event information table TBL2, the CPU 21 may automatically transmit the event information of the event information table TBL2 or the CPU 21 may transmit the event information of the event information table TBL2 immediately after the event information is recorded in the event information table TBL2.

### Event Information Display Process

According to a predetermined application program, the user can cause the display section 2a of the smartphone 2 to display event information based on the information of the event information table TBL2 received from the activity meter 1.

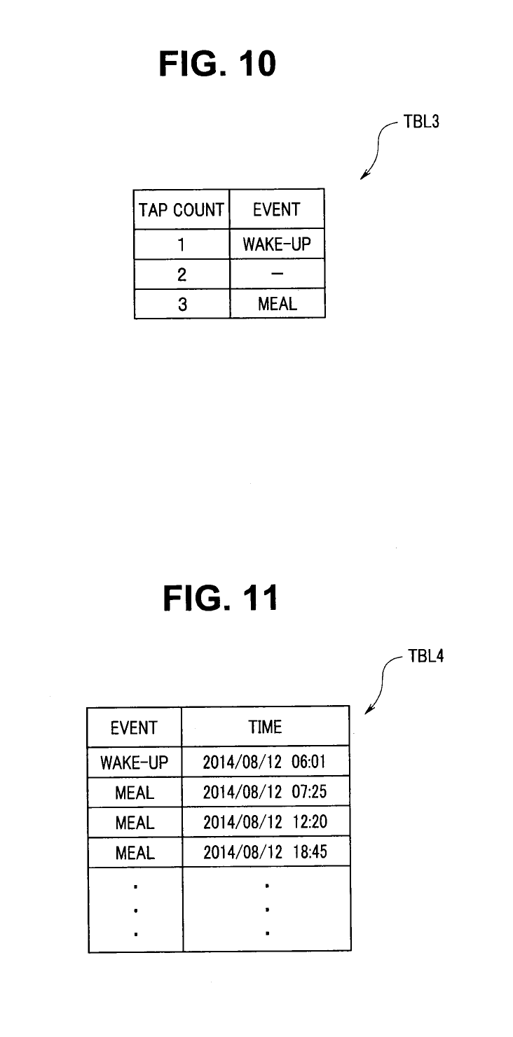

For example, a correspondence table TBL3 as shown in FIG. 10 is preset in the smartphone 2, and a CPU (not shown) of the smartphone 2 can look up the correspondence table TBL3 in FIG. 10 and display an event list as shown in FIG. 11 based on the information of the event information table TBL2.

FIG. 10 illustrates a configuration example of the correspondence table TBL3 which registers a correspondence between tap counts and events.

The correspondence table TBL3 which shows a correspondence between tap counts and events is registered in the memory of the smartphone 2. According to a predetermined application program, the user can cause contents of an event for each tap count to be registered in the correspondence table TBL3.

According to the predetermined application program of the smartphone 2, the CPU of the smartphone 2 displays event recording information as shown in FIG. 11 in a list format based on the information on the event information table TBL2 received from the activity meter 1 with reference to the correspondence table TBL3.

FIG. 11 is a display example of the event recording information list TBL4 displayed on the display section 2a. The user can check events such as the user's own action with reference to the event recording information list TBL4 displayed on the display section 2a of the smartphone 2.

As described above, the user can easily record a desired event using the activity meter 1 and cause the result thereof to be displayed on the terminal apparatus such as the smartphone as event information.

Therefore, according to the aforementioned embodiment, it is possible to provide an activity meter capable of easily recording a desired event through a simple tapping operation.

Particularly, the user needs only to tap the band 4 which is the case of the activity meter 1, and can thereby record event information through a simple operation.

Moreover, since the number of taps is not predetermined for various events and the user can arbitrarily register a tap count in association with a desired event in the activity meter 1, and the user can thereby freely record various events. Conversely, once the user arbitrarily registers a tap count in the activity meter 1, the user can also record the tap count in association with a desired event at any time.

Moreover, the activity meter 1 need not determine information on a plurality of events and information on a tapping operation corresponding to each event and set the events and the information in a memory in advance, and therefore this may lead to a reduction in the memory capacity.

Note that a tap of the aforementioned tapping operation is a single tap, but the tap may also be a double tap. In that case, the tap count is the number of double taps, and information on the tapping operation registered in the tapping operation information registration table is defined by the number of double taps.

Moreover, a tapping operation may also be one that has a difference in tapping rhythm of a single tap or double tap. In that case, information on the tapping operation registered in the tapping operation information registration table includes information on the rhythm of a single tap or double tap.

In the aforementioned embodiment, the respective processes are implemented by a software program executed by the CPU 21, but the processes may also be implemented by a hardware circuit.

Furthermore, in the aforementioned embodiment, event information is transmitted to the smartphone 2 as a terminal and the event information is displayed on the display section 2a of the smartphone 2, but the event information may also be transmitted to a terminal such as a tablet terminal or personal computer and the event information may be displayed on a display section of the tablet terminal or the like.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An activity meter comprising:
a tapping operation detection section configured to detect, when a tapping operation is performed, information on the tapping operation;
a tapping operation information registration section configured to register the information on the tapping operation based on detected information from the tapping operation detection section;
a tapping operation generation detection section configured to detect generation of a tapping operation identical to the tapping operation corresponding to the registered information from a detected signal of the tapping operation detection section; and
a time information storage section configured to store, when the tapping operation generation detection section detects generation of the tapping operation identical to the tapping operation corresponding to the registered information, time information when the identical tapping operation is generated.

2. The activity meter according to claim 1, wherein the information on the tapping operation is defined by a tap count.

3. The activity meter according to claim 1, wherein the information on the tapping operation comprises information on a rhythm of the tapping operation.

4. The activity meter according to claim 1, wherein the tapping operation information registration section registers a plurality of pieces of information on a plurality of mutually different tapping operations.

5. The activity meter according to claim 1, comprising a case configured to incorporate the tapping operation detection section,
wherein the tapping operation is performed on the case.

6. The activity meter according to claim 5, wherein the case is a wristband.

7. The activity meter according to claim 6, wherein the tapping operation detection section, the tapping operation information registration section, the tapping operation generation detection section and the time information storage section are housed in one unit, and
the unit is attachable/detachable to/from the case.

8. An activity meter comprising:
an acceleration sensor;
a tapping operation information registration section configured to register, based on an output signal of the acceleration sensor when a tapping operation is performed, information on the tapping operation;
a tapping operation generation detection section configured to detect generation of a tapping operation identical to the tapping operation corresponding to the registered information from the output signal of the acceleration sensor; and
a time information storage section configured to store, when the tapping operation generation detection section detects generation of the tapping operation identical to the tapping operation corresponding to the registered information, time information when the identical tapping operation is generated.

9. The activity meter according to claim 8, wherein the information on the tapping operation is defined by a tap count.

10. The activity meter according to claim 8, wherein the information on the tapping operation comprises information on a rhythm of the tap.

11. The activity meter according to claim 8, wherein the tapping operation information registration section registers a plurality of pieces of information on a plurality of mutually different tapping operations.

12. The activity meter according to claim 8, wherein the tapping operation information registration section registers, when a first operation is performed, the information on the tapping operation.

13. The activity meter according to claim 12, comprising an operation button,
wherein the first operation is an operation on the operation button.

14. The activity meter according to claim 12, wherein after the first operation is performed, the tapping operation information registration section registers the information on the tapping operation based on an output signal of the acceleration sensor within a first predetermined time period.

15. The activity meter according to claim 12, wherein when a second operation is performed, the tapping operation generation detection section detects generation of the tapping operation identical to the tapping operation corresponding to the registered information.

16. The activity meter according to claim 15, comprising an operation button,
wherein the second operation is an operation on the operation button.

17. The activity meter according to claim 8, comprising a case configured to incorporate the acceleration sensor,
wherein the tapping operation is performed on the case.

18. The activity meter according to claim 17, wherein the case is a wristband.

19. The activity meter according to claim 18, wherein the acceleration sensor, the tapping operation information registration section, the tapping operation generation detection section and the time information storage section are housed in one unit, and
the unit is attachable/detachable to/from the case.

20. An event information recording system comprising:
an activity meter wearable on a target person whose activity amount is measured; and
a terminal communicable with the activity meter,
wherein the activity meter comprises:
an acceleration sensor;
a tapping operation information registration section configured to register, based on an output signal of the acceleration sensor when a tapping operation is performed, information on the tapping operation;
a tapping operation generation detection section configured to detect generation of a tapping operation identical to the tapping operation corresponding to the registered information from the output signal of the acceleration sensor;
a time information storage section configured to store, when the tapping operation generation detection section detects generation of the tapping operation identical to the tapping operation corresponding to the registered information, time information when the identical tapping operation is generated; and
an information transmitting section configured to transmit the stored time information, and
the terminal comprises a storage section configured to store the time information received from the activity meter in association with predetermined event information.
